(19) United States
(12) Patent Application Publication
(10) Pub. No.: US 2024/0265270 A1
(43) Pub. Date: Aug. 8, 2024

(54) RNA LOCATION PREDICTION METHOD AND APPARATUS, AND STORAGE MEDIUM

(71) Applicant: BOE Technology Group Co., Ltd., Beijing (CN)

(72) Inventors: Yulan HU, Beijing (CN); Zhenzhong ZHANG, Beijing (CN)

(21) Appl. No.: 18/291,563

(22) PCT Filed: Oct. 29, 2021
(86) PCT No.: PCT/CN2021/127273

§ 371 Date: Jan. 23, 2024

(51) Int. Cl. G06N 3/123; G16B 5/00; G16B 40/20

(52) U.S. Cl. CPC G06N 3/123; G16B 5/00; G16B 40/20

(57) ABSTRACT

An RNA location prediction method and apparatus, and a storage medium. The method comprises: acquiring sequence feature information and structure feature information of LncRNA to be located (101); performing calculation on the sequence feature information and/or the structure feature information on the basis of an attention mechanism, to obtain an attention value of the sequence feature information and/or the structure feature information (102); and inputting the attention value into a classification prediction model to obtain a location prediction result of the LncRNA to be located (103).

### CROSS-REFERENCE TO RELATED APPLICATION

This application is a national phase entry under 35 U.S.C § 371 of International Application No. PCT/CN2021/127273, filed on Oct. 29, 2021, the entire content of which is incorporated herein by reference.

### TECHNICAL FIELD

The present disclosure relates to the field of bioinformation, in particular to an RNA location prediction method and apparatus, and a storage medium.

### BACKGROUND

Long non-coding ribonucleic acid (lncRNA) refers to an RNA molecule that does not encode proteins and has a sequence length greater than 200 bases.

Studies have demonstrated that LncRNAs play different biological functions in subcells at different locations, e.g., LncRNAs located in a nucleus typically participate in regulating gene transcription, and LncRNAs located in a cytoplasm usually post-transcriptionally regulate gene expression. In general, LncRNA subcellular location includes an experimental method and a calculation prediction method, however, it becomes increasingly important to develop an accurate and efficient calculation prediction method due to the time consuming and labor consuming, and high blindness of location of the experimental method for determining the presence of LncRNA in subcells.

At present, the existing LncRNA subcellular location calculation prediction method mainly includes following methods.

A first method is LncLocator based on an autoencoder and ensemble learning, and this method uses 3-mer to represent sequence features, utilizes a stacked autoencoder to extract high-level sequence features, and uses a support vector machine (SVM), a random forest, and stacked network ensemble learning for classification prediction; a second method is iLoc lncRNA based on a pseudo K-tuple nucleotide composition (PseKNC) feature representation, and this method extracts sequence features by combining the 8-mer with the pseudo K-tuple nucleotide composition (PseKNC), uses a binomial distribution-based feature selection method, and utilizes a support vector machine (SVM) for classification prediction; and a third method is DeepLncRNA, which is a deep learning method based on sequence features, and this method uses three types of features of the k-mer encoding, th RNA-binding protein motif features, and the genomic sites, and uses a deep neural network (DNN) for binary prediction of nuclei and cytoplasms.

Since the above methods all use a k-mer frequency encoding sequence of a sequence of LncRNA, problems such as sparseness of features, loss of sequence information of sequences in a continuous space, etc., tend to occur, resulting in poor accuracy of prediction of LncRNA subcellular location.

### SUMMARY

The present disclosure provides an RNA location prediction method and apparatus, and a storage medium to solve the above technical problems existing in the related art.

In a first aspect, in order to solve the above technical problems, the technical solution of an RNA location prediction method provided by embodiments of the present disclosure is as follows:
obtaining sequence feature information and structure feature information of LncRNA to be located;
performing a calculation on the sequence feature information and/or the structure feature information on the basis of an attention mechanism to obtain an attention value of the sequence feature information and/or the structure feature information; and
inputting the attention value into a classification prediction model to obtain a location prediction result of the LncRNA to be located.

In a possible implementation, the obtaining the sequence feature information of the LncRNA to be located includes:
performing k-mer encoding on a sequence of the LncRNA to be located to obtain at least one k-mer encoding set; wherein k-mer in each k-mer encoding set includes the same number of bases, and k-mer in different k-mer encoding sets includes different numbers of bases;
obtaining an embedding vector representation of each k-mer encoding in each k-mer encoding set based on a k-mer pre-training model; and
extracting the sequence feature information of the LncRNA to be located from all embedding vector representations with a convolutional neural network.

In a possible implementation, the performing k-mer encoding on the sequence of the LncRNA to be located to obtain a plurality of k-mer encoding sets includes:
sequentially taking continuous k number of bases starting from a first base of the sequence of the LncRNA to be located according to k corresponding to each k-mer encoding set to form one k-mer in a corresponding k-mer encoding set until the last k number of bases in the sequence of the LncRNA to be located are taken to form the corresponding k-mer encoding set; wherein first bases of two adjacent k-mers in the same k-mer encoding set are adjacent in the sequence of the LncRNA, and k is a natural number.

In a possible implementation, a training process of the k-mer pre-training model includes:
performing k-mer encoding on a sequence of each second LncRNA in a set of second LncRNAs to obtain a plurality of second k-mer encoding sets corresponding to the sequence of each second LncRNA;
taking all the second k-mer encoding sets and a plurality of special characters as a vocabulary for a BERT model; and
iteratively training the BERT model with all the second k-mer encoding sets to predict an embedding vector representation of a masked element in the second k-mer encoding sets until a value of a loss function predicted by the BERT model no longer decreases to stop training to obtain the k-mer pre-training model; the BERT model only includes a MASK-LM task in which elements in the second k-mer encoding sets are partially masked with the special characters, and different second k-mer encoding sets correspond to different masking rates, and the masking rate is the fraction of the special characters in masked second k-mer encoding.

In a possible implementation, the convolutional neural network includes:
a convolutional layer, including a plurality of convolution kernels with different sizes; and each convolution kernel is configured to perform a convolution operation on a matrix corresponding to the embedding vector representation; and
a max pooling layer connected with an output end of the convolutional layer, and configured to segment a convolution operation result output by the convolutional layer, and combine an maximum feature value in each of obtained segments into the sequence feature information.

In a possible implementation, the obtaining the structure feature information of the LncRNA to be located includes:
converting a secondary structure of the LncRNA to be located into a tree structure; and
extracting tree structure features from the tree structure with Tree Lstm as the structure feature information of the LncRNA to be located.

In a possible implementation, the converting the secondary structure of the LncRNA to be located into the tree structure includes:
taking a base pair in which bases are complementarily paired as a root node of the tree structure, and taking unpaired bases as leaf nodes of a previous node in the tree structure according to a pairing relationship of bases in the secondary structure starting from a first base of the sequence of the LncRNA to be located until the last base of the sequence of the LncRNA to be located to obtain the tree structure; and when the first base is unpaired, the root node of the tree structure is empty.

In a possible implementation, the extracting the tree structure features from the tree structure with the Tree Lstm includes:
taking outputs of all child nodes of a current node currently being processed in the tree structure as an input of the current node starting from a leaf node of the tree structure, and updating a gating vector and a memory unit corresponding to the current node according to states of the child nodes until the current node is a root node of the tree structure; input of the leaf node is a corresponding base; and
taking output of the root node as the tree structure features.

In a possible implementation, the performing the calculation on the sequence feature information and/or the structure feature information on the basis of the attention mechanism to obtain the attention value of the sequence feature information and/or the structure feature information includes:
calculating a relatedness value of a value of each dimension in the sequence feature information with the structure feature information;
performing a normalization calculation on the relatedness value corresponding to each dimension in the sequence feature information to obtain a first attention weight of the value of each dimension in the sequence feature information; and
performing a sum operation on a product of each first attention weight and the structure feature information based on the attention mechanism to obtain an attention value of the sequence feature information relative to the structure feature information.

In a possible implementation, the performing the calculation on the sequence feature information and/or the structure feature information on the basis of the attention mechanism to obtain the attention value of the sequence feature information and/or the structure feature information includes:
calculating a relatedness value of a value of each dimension in the structure feature information with the sequence feature information;
performing a normalization calculation on the relatedness value corresponding to each dimension in the structure feature information to obtain a second attention weight of the value of each dimension in the structure feature information; and
performing a sum operation on a product of each second attention weight and the sequence feature information based on the attention mechanism to obtain an attention value of the structure feature information relative to the sequence feature information.

In a possible implementation, the inputting the attention value into the classification prediction model to obtain the location prediction result of the LncRNA to be located includes:
inputting the attention value into the classification prediction model to obtain a location prediction value of the LncRNA to be located; and
taking a subcell corresponding to a maximum probability in the location prediction value as a subcell in which the LncRNA to be located is located to obtain the location prediction result.

In a possible implementation, a training process of the classification prediction model includes:
obtaining first sequence feature information and first structure feature information of each first LncRNA in a tagged set of first LncRNAs;
performing a calculation on the first sequence feature information and the first structure feature information of each first LncRNA based on an attention mechanism to obtain a first attention value;
inputting the first attention value into a classification prediction model to obtain a first location prediction value of a corresponding first LncRNA; and
calculating a loss value based on the first location prediction value and a tag value of the corresponding first LncRNA, and adjusting parameters in the classification prediction model by a back propagation algorithm until the loss value reaches a preset condition to obtain a trained classification prediction model.

In a second aspect, the embodiments of the present disclosure further provide an RNA location prediction apparatus, including:
at least one processor, and
a memory connected with the at least one processor;
the memory stores instructions which can be executed by the at least one processor, and the at least one processor performs the method in the first aspect by executing the instructions stored in the memory.

In a third aspect, the embodiments of the present disclosure further provide a readable storage medium, including:
a memory;
the memory is configured to store instructions that, when the instructions are executed by a processor, cause an apparatus including the readable storage medium to perform the method in the first aspect.

### BRIEF DESCRIPTION OF FIGURES

FIG. 1 is a flowchart of an RNA location prediction method according to an embodiment of the present disclosure.

FIG. 2 is a schematic diagram of performing k-mer encoding on a sequence of LncRNA to be located according to an embodiment of the present disclosure.

FIG. 3 is another schematic diagram of performing k-mer encoding on a sequence of the LncRNA to be located according to an embodiment of the present disclosure.

FIG. 4 is a schematic diagram of obtaining an Embedding representation of a k-mer encoding set according to an embodiment of the present disclosure.

FIG. 5 is a schematic diagram of obtaining sequence feature information of the LncRNA to be located according to an embodiment of the present disclosure.

FIG. 6 is a schematic diagram of converting a secondary structure of the LncRNA to be located into a tree structure according to an embodiment of the present disclosure.

FIG. 7 is a structural schematic diagram of Tree LSTM according to an embodiment of the present disclosure.

FIG. 8 is a schematic diagram of obtaining a predicted value of the sequence of the LncRNA to be located according to an embodiment of the present disclosure.

### DETAILED DESCRIPTION

Embodiments of the present disclosure provide an RNA location prediction method and apparatus, and a storage medium to solve the above technical problems existing in the related art.

In order to better understand the above technical solutions, the technical solutions of the present disclosure will be described in detail below with reference to the drawings and specific embodiments. It should be understood that the embodiments of the present disclosure and the specific features in the embodiments are detailed descriptions of the technical solutions of the present disclosure, rather than limitations of the technical solutions of the present disclosure. In the case of no conflict, the embodiments of the present disclosure and the technical features in the embodiments can be combined with each other.

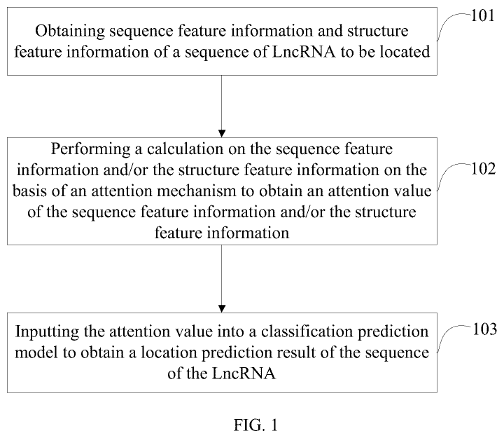

Please refer to FIG. 1, the embodiments of the present disclosure provide an RNA location prediction method, and a processing process of the method is as follows.

Step 101: obtaining sequence feature information and structure feature information of LncRNA to be located.

LncRNAs may be distributed in subcells such as a cytoplasm, a nucleus, a chromatin, a nucleolus, a mitochondrion, and the like, and LncRNAs located in different subcells may play different biological functions, such as LncRNAs in the nucleus typically participate in regulating gene transcription, and LncRNAs located in the cytoplasm typically post-transcriptionally regulate gene expression.

A sequence of the LncRNA refers to the arrangement of bases in the LncRNA, a biologist can manually obtain the sequence feature information and the structure feature information of the LncRNA to be located, and in order to improve the work efficiency, the present disclosure also provides the following manner for obtaining the sequence feature information and the structure feature information of the LncRNA to be located.

Obtaining the sequence feature information of the LncRNA to be located can be achieved by:
performing k-mer encoding on a sequence of the LncRNA to be located to obtain at least one k-mer encoding set; wherein k-mer in each k-mer encoding set includes the same number of bases, and k-mer in different k-mer encoding sets includes different numbers of bases; obtaining an embedding vector representation for each k-mer encoding in each k-mer encoding set based on a k-mer pre-training model; and extracting the sequence feature information of the LncRNA to be located from all embedding vector representations with a convolutional neural network.

The performing the k-mer encoding on the sequence of the LncRNA to be located to obtain the plurality of k-mer encoding sets can be achieved by:
sequentially taking continuous k number of bases starting from a first base of the sequence of the LncRNA to be located according to k corresponding to each k-mer encoding set to form one k-mer in the corresponding k-mer encoding set until last k number of bases in the sequence of the LncRNA to be located are taken to form the corresponding k-mer encoding set; and the first bases of two adjacent k-mers in the same k-mer encoding set are adjacent in the sequence of the LncRNA, and k is a natural number.

For example, a sequence of LncRNA to be located with a sequence length of L is R=R₁R₂R₃ . . . R_{L}, wherein R_{i′}∈(A, U, G, C), i′ is in the range of 1 to L, R_{i′} represents an i′-th base in the sequence of the LncRNA to be located, L is a natural number, and A, U, G and C represent bases in the LncRNA.

If k=1, then one base is taken starting from R₁ (i.e., one base corresponding to R₁), then one base corresponding to R₂ is taken, . . . , until a last base (i.e., a base corresponding to R_{L}) is taken to form a 1-mer encoding set that can be expressed as R₁′=[R₁, R₂, R₃, . . . , R_{L}], with a total of L elements.

If k=2, then two bases are consecutively taken in an overlapping manner starting from R₁ until all bases are taken, e.g., two bases corresponding to R₁R₂ are first taken, then two bases are taken starting from R₂ (i.e., two bases corresponding to R₂R₃), . . . , until the last two bases are taken (i.e., bases corresponding to R_{L-1}R_{L}) to form a 2-mer encoding set which can be expressed as R₂′=[R₁R₂, R₂R₃, . . . , R_{L-1}R_{L}], with a total of L−1 elements; or, two bases can be consecutively taken in a non-overlapping manner starting from R₁ until all bases are taken, e.g., two bases corresponding to R₁R₂ are first taken, then two bases corresponding to R₃R₄ are taken, . . . , until the last two bases (i.e., two bases corresponding to R_{L-1}R_{L}) are taken to form a 2-mer encoding set which can be expressed as R₂′=[R₁R₂, R₃R₄, . . . , R_{L-1}R_{L}], with a total of L/2 elements.

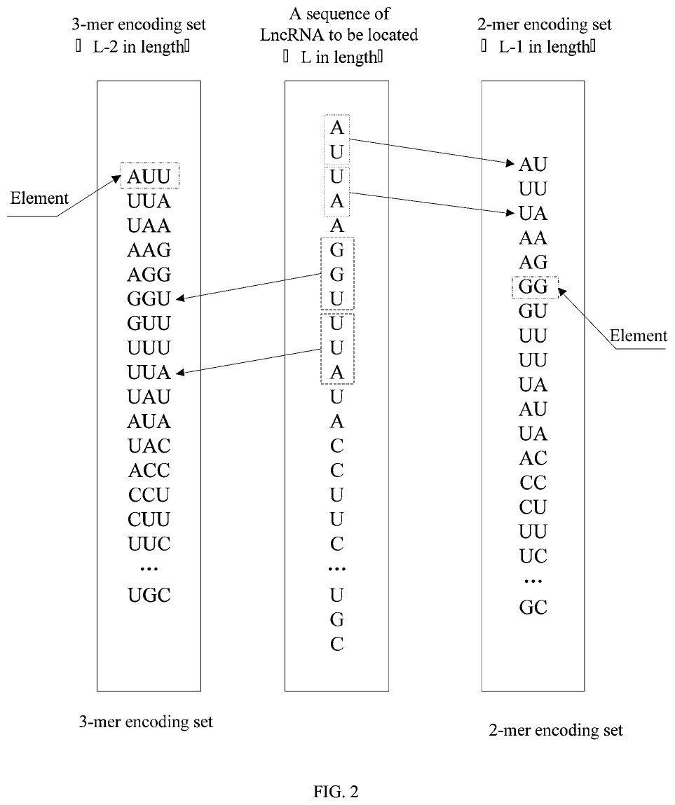

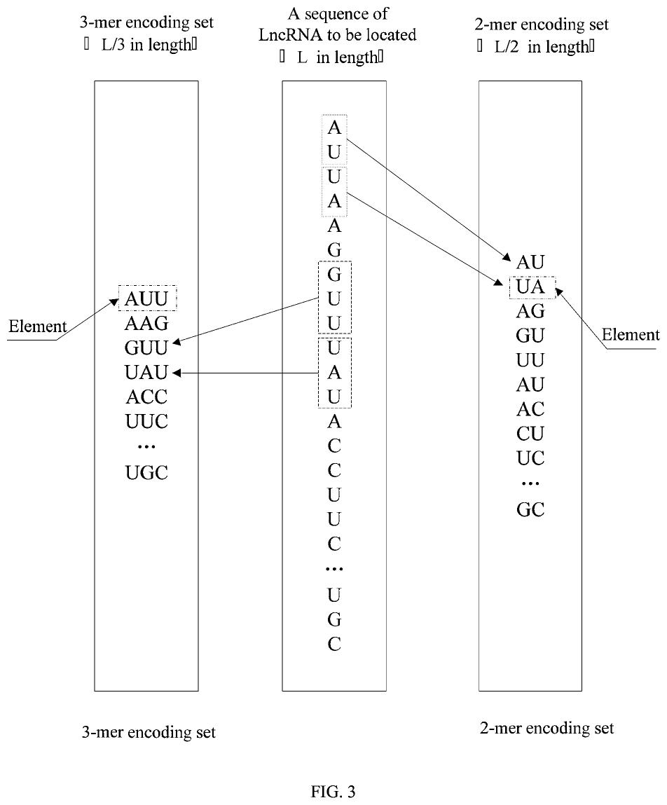

If k=3, then three bases are consecutively taken in an overlapping manner starting from R₁ until all bases are taken, e.g., three bases corresponding to R₁R₂R₃ are first taken, then three bases are taken starting from R₂ (i.e., three bases corresponding to R₂R₃R₄), . . . , until the last three bases are taken (i.e., bases corresponding to R_{L-2}R_{L-1}R_{L}) to form a 3-mer encoding set which can be expressed as R₃′=[R₁R₂R₃, R₂R₃R₄, . . . , R_{L-2}R_{L-1}R_{L}], with a total of L−2 elements, and as shown in FIG. 2, a schematic diagram of performing k-mer encoding on a sequence of LncRNA to be located according to an embodiment of the present disclosure is shown. Or, three bases may be consecutively taken in a non-overlapping manner starting from R₁ until all bases are taken, e.g., three bases corresponding to R₁R₂R₃ are first taken, then three bases corresponding to R₄R₅R₆ are taken, . . . , until last three bases (i.e., three bases corresponding to R_{L-2}R_{L-1}R_{L}) are taken to form a 3-mer encoding set which can be expressed as R₃′=[R₁R₂R₃, R₄R₅R₆, . . . , R_{L-2}R_{L-1}R_{L}], with a total of L/3 elements, and as shown in FIG. 3, another schematic diagram of performing k-mer coding on the sequence of the LncRNA to be located according to an embodiment of the present disclosure is shown.

If k=4, then four bases are consecutively taken in an overlapping manner starting from R₁ until all bases are taken, e.g., four bases corresponding to R₁R₂R₃R₄ are first taken, then four bases are taken starting from R₂ (i.e., four bases corresponding to R₂R₃R₄R₅), . . . , until the last four bases are taken (i.e., bases corresponding to R_{L-3}R_{L-2}R_{L-1}R_{L}) to form a 4-mer encoding set which can be expressed as R₄′=[R₁R₂R₃R₄, R₂R₃R₄R₅, . . . , R_{L-3}R_{L-2}R_{L-1}R_{L}], with a total of L−3 elements; or, four bases may be consecutively taken in a non-overlapping manner starting from R₁ until all bases are taken, e.g., four bases corresponding to R₁R₂R₃R₄ are first taken, then four bases corresponding to R₅R₆R₇R₈ are taken, . . . , until the last four bases are taken (i.e., bases corresponding to R_{L-3}R_{L-2}R_{L-1}R_{L}) to form a 4-mer encoding set which can be expressed as R₄′=[R₁R₂R₃R₄, R₅R₆R₇R₈, . . . , R_{L-3}R_{L-2}R_{L-1}R_{L}], with a total of L/4 elements.

It needs to be understood that the length of the sequence of the LncRNA to be located is the number of bases included in the sequence, each element in the k-mer encoding set is a fragment in the sequence of the LncRNA to be located, the length of the k-mer encoding set is the number of elements included in the k-mer encoding set, and when k>1, when bases in the sequence of the LncRNA are taken in an overlapping manner, the number of overlapping bases in two adjacent fragments can be determined according to actual needs, which is not limited to the above examples.

By simultaneously performing k-mer encoding with different k values on the sequence of the LncRNA to be located, the problem of sequence information loss in a continuous space caused by sparse sequence features when single k-mer coding is performed can be prevented, thereby improving the accuracy of LncRNA subcellular location prediction.

After the plurality of the k-mer encoding sets of the sequence of the LncRNA to be located are obtained, the k-mer encoding sets are input into the k-mer pre-training model to obtain the embedding vector representations corresponding to the k-mer encoding sets, and then the sequence feature information of the LncRNA to be located is extracted from all the embedding vector representations with the convolutional neural network.

A training process of the above k-mer pre-training model includes:
performing k-mer encoding on a sequence of each second LncRNA in a set of second LncRNAs to obtain a plurality of second k-mer encoding sets corresponding to the sequence of each second LncRNA; taking all the second k-mer encoding sets and a plurality of special characters as a vocabulary for a BERT model; and iteratively training the BERT model with all the second k-mer encoding sets to predict embedding vector representations of masked elements in the second k-mer encoding sets until a value of a loss function of the BERT model no longer decreases to stop training, so as to obtain the k-mer pre-training model. The BERT model only includes a MASK-LM task in which elements in the second k-mer encoding sets are partially masked with the special characters, and different second k-mer encoding sets correspond to different masking rates, and the masking rate is the fraction of the special characters in masked second k-mer encoding.

The second LncRNA in the set of second LncRNAs can be untagged historical LncRNAs (i.e., historical LncRNAs that are not tagged with subcellular locations) collected from an RNAcentral database.

After the set of second LncRNAs is obtained, k-mer encoding is performed on the sequence of each second LncRNA in the set of second LncRNAs to obtain the plurality of the second k-mer encoding sets corresponding to the sequence of each second LncRNA; in particular, a manner of performing k-mer encoding on the sequence of each second LncRNA may be the same as the manner of performing k-mer encoding on the sequence of the LncRNA to be located, and k is in the same range. If 1-mer encoding to 4-mer encoding need to be performed on the sequence of the LncRNA to be located, 1-mer encoding to 4-mer encoding also need to be performed on each second LncRNA, which will not be repeated.

All the second k-mer encoding sets and the plurality of the special characters are taken as the vocabulary (also referred to as a k-mer vocabulary) for the BERT model, the special characters include ‘<pad>’, ‘<mask>’, ‘<cls>’, ‘<sep>’, and ‘<unk>’.

In general, a traditional BERT model includes two pre-training tasks: Masked Language Model (Masked LM) and Next Sentence Prediction. The task of Masked LM is described as: a sentence is given, one or several words in the sentence are randomly erased, and what the erased words are is required to be predicted based on the remaining vocabularies. For example, 15% of vocabularies in a sentence are randomly selected for prediction, vocabularies that are erased in the original sentence are replaced with one special symbol [MASK] in 80% of cases, and are replaced with an arbitrary word in 10% of cases, and the original vocabularies are kept unchanged in the remaining 10% of cases, so that when a vocabulary is predicted, the BERT model does not know whether a vocabulary input at the corresponding position is a correct vocabulary (10% probability), which forces the BERT model to rely more on context information to predict the vocabulary and endows the BERT model with a certain error correction capability. While the task of Next Sentence Prediction is described as: two sentences in an article are given, it is determined that whether a second sentence immediately follows a first sentence in a text, and in the actual pre-training process, 50% correct sentence pairs and 50% incorrect sentence pairs are randomly selected from a text corpus for training. By combining the Next Sentence Prediction task with the Masked LM task, the BERT model is able to more accurately depict semantic information at a sentence level and even a textual level.

The BERT model in the present disclosure is a BERT model only containing the MASK-LM task, i.e., a module corresponding to the Next Sentence Prediction task in the conventional BERT model is eliminated (which can reduce the training complexity), and elements in the second k-mer encoding sets are partially masked by using the plurality of the special characters in the MASK-LM task, and different second k-mer encoding sets correspond to different masking rates, and the masking rate is the fraction of special characters in the masked second k-mer encoding. For example, when the embedding vector representation corresponding to the 1-mer encoding set is trained, 40% of 1-mers, 20% of 2-mers, 20% of 3-mers and 20% of 4-kmers are masked during masking.

Since the BERT model in the present disclosure uses the plurality of the second k-mer encoding sets as the k-mer vocabulary, its semantic information is richer, and since a plurality of special characters are added in the MASK-LM task, the prediction ability of the BERT model can be improved.

In the embodiments provided by the present disclosure, the BERT model is composed of a plurality of Transformer structural units, a plurality of multi-headed attentions, a hidden layer, etc. The number of nodes of the hidden layer is an integer multiple of the total number of the multi-headed attentions. For example, the BERT model uses 12 Transformer structural blocks, 12 multi-headed attentions, 768 nodes of the hidden layer, and 6149 position vector Embedding dimensions.

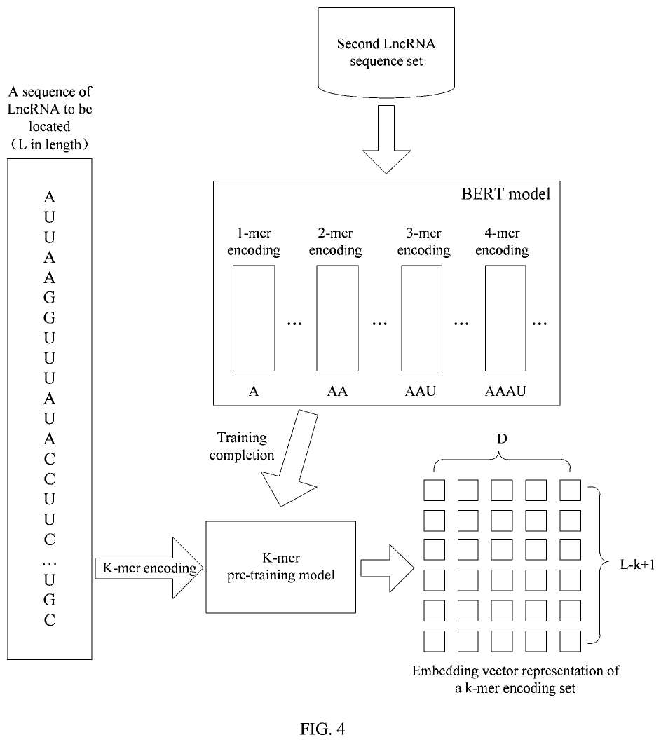

After constructing the BERT model only containing the MASK-LM task, and preparing the second k-mer encoding sets corresponding to the set of second LncRNAs, the BERT model is iteratively trained with the plurality of the second k-mer encoding sets corresponding to each second LncRNA to predict embedding vector representations of masked elements in the second k-mer encoding sets until the value of the loss function of the BERT model no longer decreases to stop training, so as to obtain the k-mer pre-training model. In this way, the k-mer pre-training model can be used to obtain the embedding vector representations of the k-mer encoding sets of the LncRNA to be located. If the dimension of the k-mer pre-training model is D and k is 1-4, the embedding vector representations corresponding to the 1-mer encoding set to the 4-mer encoding set of the sequence of the LncRNA to be located can be represented in sequence by the following matrixes: L×D, (L−1)×D, (L−2)×D, and (L−3)×D, and L is the length of the sequence of the LncRNA to be located. As shown in FIG. 4, a schematic diagram of obtaining an embedding vector representation of a k-mer encoding set according to the embodiments of the present disclosure is shown.

After the embedding vector representations of the k-mer encoding sets of the sequence of the LncRNA to be located are obtained, the sequence feature information of the LncRNA to be located can be extracted from all the embedding vector representations with the convolutional neural network.

The above convolutional neural network includes:
a convolutional layer, including a plurality of convolution kernels with different sizes, and each convolution kernel is configured to perform a convolution operation on a matrix corresponding to the embedding vector representation; and
a max pooling layer connected with an output end of the convolutional layer, and configured to segment a convolution operation result output by the convolutional layer, and combine an maximum feature value in each of obtained segments into the sequence feature information.

For example, three convolution kernels may be included in the convolutional layer, a size h of the three convolution kernels is equal to 3, 4, and 5, respectively. Accordingly, a dimension of the three convolution kernels is 3×D, 4×D, and 5×D, respectively. Convolution is performed by using the three convolution kernels and a matrix corresponding to each embedding vector representation to obtain a convolution result corresponding to each embedding vector representation, which is a matrix of (n−h+1)×1×m, wherein n is the total number of elements in the k-mer encoding sets corresponding to the embedding vector representations, h is a size of each convolution kernel, and m is the total number of the convolution kernels in the convolutional layer.

A convolution calculation formula is:

$${C}_{{k}^{′}} = g ( {wx}_{{k}^{′} : {k}^{′} + h - 1} + b ) .$$

Here, Ck′ is an output result of the k′-th convolution operation of the convolution kernel of h, k′=1−n, g ( ) is an activation function, w is the convolution kernel, b is a bias of the convolution kernel, and x_{k′:k′+h-1} denotes elements in the k′-th row to the (k′+h−1)-th row in the matrix corresponding to each embedding vector representation of a k′-mer encoding set. The embedding vector representations of each k-mer encoding set can be calculated by using the convolution calculation formula, and then all convolution operation results (denoted as C, i.e., the convolution operation results output by the convolutional layer, C=(C₁, C₂, . . . , Cₙ)) for the embedding vector representations corresponding to the LncRNA to be located are obtained.

Then the convolution operation result output by the convolutional layer is segmented with the max pooling layer, and a maximum feature value in each segment is obtained (equivalent to extracting a primary feature), and these maximum feature values are combined into the sequence feature information of the LncRNA to be located (equivalent to extracting a high-level feature).

A pooling calculation formula employed by the max pooling layer:

$$P = ( max {C}_{1 : q} , … , max {C}_{n - q + 1 : n} ) .$$

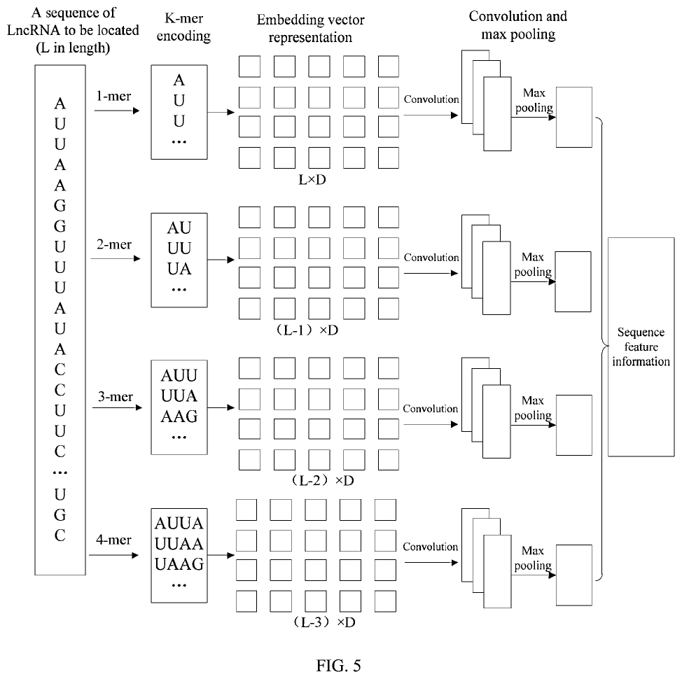

Here, P is the sequence feature information of the LncRNA to be located, q=n/p, p is the number of segments, C_{1:q} is the first row to the q-th row of a matrix corresponding to the convolution result output by the convolutional layer, C_{n-q+1:n} is the n-th row to the (n−q+1)-th row of the matrix corresponding to the convolution result output by the convolutional layer, maxC_{1:q} is a maximum feature taken from C_{1:q}, and maxC_{n-q+1:n} is a maximum feature taken from C_{n-q+1:n}. Please refer to FIG. 5, which is a schematic diagram of obtaining sequence feature information of LncRNA to be located according to the embodiments of the present disclosure.

The sequence feature information under different k-mer conditions is extracted by the convolutional neural network, and since the convolutional neural network can perform parallel computing, sequence feature information of different scales can be rapidly captured.

Obtaining the structure feature information of the LncRNA to be located can be achieved by:
converting a secondary structure of the LncRNA to be located into a tree structure; and extracting a tree structure feature from the tree structure with Tree Lstm as the structure feature information of the LncRNA to be located.

The secondary structure of the LncRNA to be located can be represented by a plane figure, a secondary structure plan text (a CT file), a dot-bracket notation, and the like. A core idea of the dot-bracket notation is to use “(“and”)” to represent complementary pairing of two bases, and use “.” to represent unpaired bases. The secondary structure of the LncRNA to be located can be calculated by a program or obtained by other means such as database search and experimental validation.

Taking a sequence “AGUGAAGGCACAAGCCUUAC” of the LncRNA to be located as an example, its secondary structure can be represented as: “.((((.(((. . . .)))))))” by the dot-bracket notation.

After the secondary structure of the LncRNA to be located is obtained, converting the secondary structure into the tree structure can be specifically achieved by:
taking a base pair in which bases are complementarily paired as a root node of the tree structure, and taking unpaired bases as leaf nodes of a previous node in the tree structure according to a pairing relationship of bases in the secondary structure starting from a first base of the sequence of the LncRNA to be located until the last base of the sequence of the LncRNA to be located to obtain the tree structure; wherein when the first base is unpaired, the root node of the tree structure is empty.

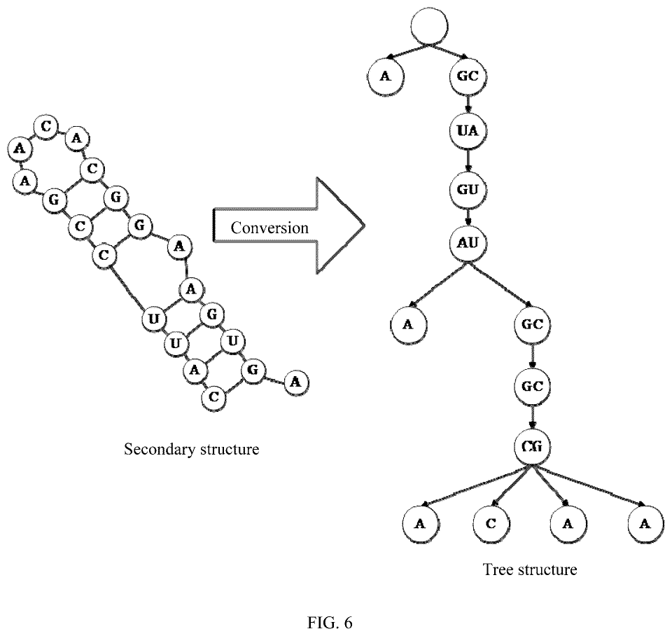

Please refer to FIG. 6, which is a schematic diagram of converting a secondary structure of LncRNA to be located into a tree structure according to the embodiments of the present disclosure.

A sequence of the LncRNA to be located in FIG. 6 is: “AGUGAAGGCACAAGCCUUAC”, the secondary structure is represented as: “.((((.(((. . . .)))))))” by the dot-bracket notation, since A is unpaired, a root node is empty, A and G are leaves of the empty root node, since G has a complementarily paired base (C), a base pair (GC) is taken as a root node of a next base (U), and since a base U is complementarily paired with a base A, a base pair UA is taken as a root node of a next base until the last base (C) of the sequence of the LncRNA to be located to obtain a tree structure as shown in FIG. 6. The tree structure described above may be stored in a level order of nodes in the tree structure.

After the tree structure of the LncRNA to be located is obtained, the tree structure features can be extracted from the tree structure of the LncRNA to be located with the Tree Lstm, and used as the structure feature information of the LncRNA to be located.

Extracting the tree structure features from the tree structure with the Tree Lstm can be achieved by:
taking outputs of all child nodes of a current node currently being processed in the tree structure as an input of the current node starting from a leaf node of the tree structure, and updating a gating vector and a memory unit corresponding to the current node according to states of the child nodes until the current node is a root node of the tree structure; wherein input of the leaf node is a corresponding base; and taking output of the root node as the tree structure features.

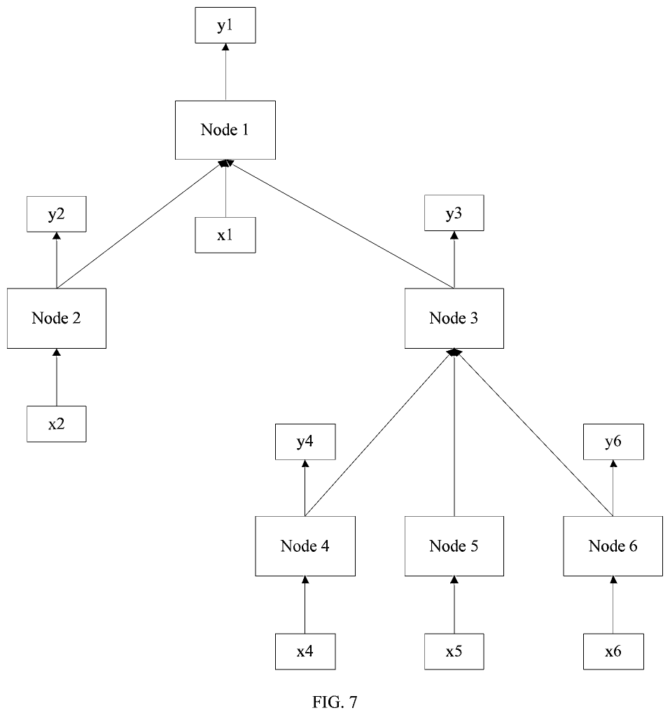

Please refer to FIG. 7, which is a structural schematic diagram of Tree LSTM according to the embodiments of the present disclosure. In FIG. 7, a node 2, a node 4, a node 5, and a node 6 are leaf nodes, the node 4 to the node 6 are child nodes of a node 3, the node 2 and the node 3 are child nodes of a node 1, and the node 1 is a root node; x1-x6 are inputs of corresponding nodes in the tree, y1-y6 are outputs of corresponding nodes in the tree, taking the condition that the node 3 in FIG. 7 is taken as a current node as an example, outputs (y4-y6) of all child nodes (the node 4 to the node 6) of the node 3 are taken as an input of the node 3, and a gating vector and a memory unit of the node 3 are updated according to states of the node 4 to the node 6; and similarly, a gating vector and a memory unit of the node 1 can be updated, and finally output of the node 1 is obtained as tree structure features of a tree structure shown in FIG. 7.

The above process is expressed by a Tree LSTM formula as follows:

$${\tilde{h}}_{j} = {\sum }_{k ∈ C ( j )} {h}_{k} ; \\ {i}_{j} = σ ( {W}^{( j )} {x}_{j} + {U}^{( j )} {\tilde{h}}_{j} + {b}^{( j )} ) ; \\ {f}_{j k} = σ ( {W}^{( f )} {x}_{j} + {U}^{( f )} {\tilde{h}}_{k} + {b}^{( f )} ) k ∈ C ( j ) ; \\ {o}_{j} = σ ( {W}^{( o )} {x}_{j} + {U}^{( o )} {\tilde{h}}_{j} + {b}^{( o )} ) ; \\ {u}_{j} = tanh ( {W}^{( u )} {x}_{j} + {U}^{( u )} {\tilde{h}}_{j} + {b}^{( u )} ) ; \\ {c}_{j} = {i}_{j} ⊙ {u}_{j} + {\sum }_{k ∈ C ( j )} {f}_{j k} {c}_{k} ; \\ {h}_{j} = {o}_{j} ⊙ tanh ( {c}_{j} ) ;$$

Here, j is a current node in a tree, and C(j) represents a set of child nodes of the current node j. xⱼ is an input vector of the current node j.

The iⱼ is an input gate of the current node j, f_{Jk} is a forget gate of each child node k of the current node j, oⱼ is an output gate of the current node j, cⱼ is a memory unit of the current node j, hⱼ is a state of a hidden layer of the current node j, and uⱼ is a temporary memory unit of the current node j; W⁽ⁱ⁾, W^{(f)}, W^{(o)}, W^{(u)}, U⁽ⁱ⁾, U^{(f)}, U^{(o)}, and U^{(u)} are weight matrixes; b⁽ⁱ⁾, b^{(f)}, b^{(o)}, and b^{(u)} are bias vectors; hⱼ is the sum of hidden layers of child nodes of the current node j; δ is a sigmoid function; and ⊙ denotes a multiplication operation (multiplication by elements).

The input of the Tree-LSTM is a plurality of child nodes, and the output of the Tree-LSTM is a parent node generated after encoding the child nodes, and a dimension of the parent node is the same as that of each child node. That is, starting from a bottommost level of a tree, a vector generated after encoding child nodes located at the same level is taken as an input of the corresponding parent node, until a root node at a topmost level of the tree is processed, and an output of the root node is taken as tree structure features of the entire tree, and taken as the structure feature information of the LncRNA to be located.

After the sequence feature information and the structure feature information of the LncRNA to be located are obtained, Step 102 may be performed.

Step 102: performing a calculation on the sequence feature information and/or the structure feature information on the basis of an attention mechanism to obtain an attention value of the sequence feature information and/or the structure feature information.

Performing an attention calculation on the sequence feature information and/or the structure feature information on the basis of the attention mechanism to obtain the attention value of the sequence feature information and/or the structure feature information can be achieved by the following two ways.

First way: a relatedness value of a value of each dimension in the sequence feature information with the structure feature information is calculated; a normalization calculation is performed on the relatedness value corresponding to each dimension in the sequence feature information to obtain a first attention weight of the value of each dimension in the sequence feature information; and a sum operation is performed on a product of each first attention weight and the structure feature information based on the attention mechanism, to obtain an attention value of the sequence feature information relative to the structure feature information.

Second way: a relatedness value of a value of each dimension in the structure feature information with the sequence feature information is calculated; a normalization calculation is performed on the relatedness value corresponding to each dimension in the structure feature information to obtain a second attention weight of the value of each dimension in the structure feature information; and a sum operation is performed on a product of each second attention weight and the sequence feature information based on the attention mechanism, to obtain an attention value of the structure feature information relative to the sequence feature information.

For example, it is assumed that the sequence feature information of the LncRNA to be located is A=[a₁, a₂, . . . , aₛ], where A is a multi-dimensional vector, s is a dimension of the multi-dimensional vector A, and a₁, a₂, and aₛ represent values of corresponding dimensions in the multi-dimensional vector A, respectively; and the structure feature information of the LncRNA to be located is B=[b₁, b₂, . . . , bₜ], where B is a multi-dimensional vector, t is a dimension of the multi-dimensional vector B, and b₁, b₂, and bₜ represent values of corresponding dimensions in the multi-dimensional vector B, respectively, and A can be regarded as a tensor of 1×s and B can be regarded as a tensor of 1×t.

Q=B, K=V=A, i.e., query=B, and key and value are X, where Q, K, V, query, key and value are parameters in a transformer, and the attention value is calculated as follows:

$${α}_{{j}^{″}} = soft max ( f ( {Q}_{{j}^{″}} , K ) ) = soft max ( {B}_{{j}^{″}} WA ) = \frac{exp ( {B}_{{j}^{″}} WA )}{{\sum }_{{j}^{′} = 1}^{w} {exp}^{{B}_{{j}^{′}} WA}} {i}^{″} = 1 ∼ t ;$$

where α_{i′} is an attention weight, Softmax ( ) is a normalization function, f is a function for calculating a correlation between Q_{i″} and K, f(Q_{i″}, . . . , K)=B_{i″}WA, and i″=1−t.

Q_{i″}=B_{i″}=b_{i″}, and an initialization value of W is a random numerical value.

The calculated weights and the corresponding key values are subjected to weighted summation to obtain the attention value of the structure feature information relative to the sequence structure information:

$$Attention ( Q , K , V ) = {\sum }_{{i}^{″} = 1}^{m} {α}_{{i}^{″}} V = {\sum }_{{i}^{″} = 1}^{m} {α}_{{i}^{″}} A ;$$

Attention (Q, K, V) obtained above is a tensor of 1× s.

Similarly, the attention value of the sequence feature information relative to the structure feature information can also be calculated: at this time, Q=A, K=V=B, i.e., query=A, key and value are B, and the obtained attention is a tensor of 1*t.

After the attention value of the sequence feature information and/or the structure feature information of the LncRNA to be located is obtained in the above manner, Step 103 may be performed.

Step 103: inputting the attention value into a classification prediction model to obtain a location prediction result of a sequence of the LncRNA.

The classification prediction model needs to be first obtained by training before the attention value of the sequence feature information and/or the structure feature information of the LncRNA to be located is input into the classification prediction model, and a specific training process of the classification prediction model is as follows.

First sequence feature information and first structure feature information of each first LncRNA in a tagged set of first LncRNAs are obtained; a calculation is performed on the first sequence feature information and the first structure feature information of each first LncRNA based on an attention mechanism to obtain a first attention value; the first attention value is input into a classification prediction model to obtain a first location prediction value of the corresponding first LncRNA; and a loss value is calculated based on the first location prediction value and a tag value of the corresponding first LncRNA, and parameters in the classification prediction model are adjusted by a back propagation algorithm until the loss value reaches a preset condition to obtain a trained classification prediction model. The above preset condition may be that a value of a loss function of a validation set no longer decreases, or, an accuracy rate of a training set or a validation set no longer improves.

The first LncRNAs in the set of first LncRNAs can be tagged historical LncRNAs collected from an RNALocate database (i.e., the subcellular locations of the historical LncRNAs are tagged), the RNALocate database is a special database for RNA subcellular location, and the latest version of this database, RNALocatev2.0, has recorded more than 210,000 RNA-related subcellular location bar catalogues and experimental data, involving more than 110,000 RNAs, including 171 subcellular locations of 104 species. Currently, LncRNA data, i.e., 9587 historical LncRNAs can be extracted from the RNALocate database, and after removal of duplicates, 6897 different historical LncRNAs are obtained to form the set of first LncRNAs of the present disclosure, with locations distributed in 40 different subcells, including a cytoplasm, a nucleus, a chromatin, a nucleolus, a mitochondrion, etc.

After the above set of first LncRNAs is obtained, the first sequence feature information and the first structure feature information of each first LncRNA in the set of first LncRNAs are obtained in the same manner as that of obtaining the sequence feature information and the structure feature information of the LncRNA to be located, which will not be repeated. Then, a calculation is performed on the first sequence feature information and the first structure feature information of each first LncRNA based on the attention mechanism to obtain the corresponding first attention value, and all the first attention values are divided into a training set and a validation set; and multilayer perceptron (MLP) is trained with the training set, the trained MLP is validated with the validation set until a validation result reaches a preset condition to obtain the classification prediction model.

Training parameters for the multilayer perceptron include: SGD which can be used for an optimizer, a batch size which can be set to be 32, dropout which can be set to be 0.001, the number of iterations (epoch) which can be set to be 100, and a dimension of an embedding vector which can be set to be 768.

After the classification prediction model is obtained, the performance of the classification prediction model can also be evaluated by using 5-fold cross-validation, and performance metrics evaluated by the 5-fold cross-validation include accuracy (ACC), sensitivity (Sn), specificity (Sp), precision (Pre), F1-score, a Matthews correlation coefficient (MCC), and an area under a receiver operating characteristic curve (ROC) (Area under Curve, AUC).

When the performance of the classification prediction model meets the requirements, the attention value corresponding to the LncRNA to be located can be input into the classification prediction model to obtain the prediction result of the LncRNA to be located, which can be specifically achieved by:
inputting the attention value into the classification prediction model to obtain a location prediction value of the LncRNA to be located; and taking a subcell corresponding to a maximum probability in the location prediction value as a subcell in which the LncRNA to be located is located to obtain the location prediction result.

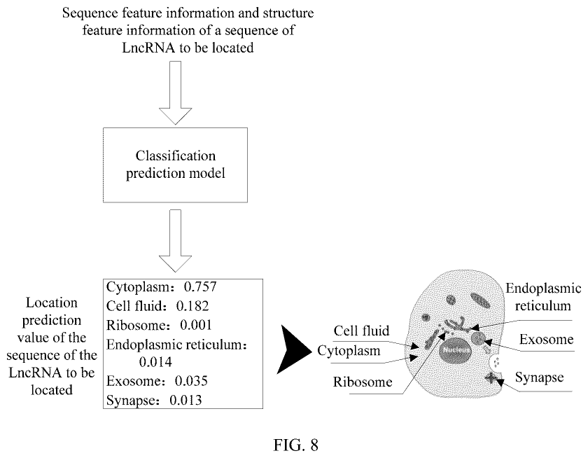

As shown in FIG. 8, a schematic diagram of obtaining a predicted value of LncRNA to be located according to the embodiments of the present disclosure is shown.

A sequence of the LncRNA to be located in FIG. 8 is AGUGAAGGCACAAGCCUUAC, the secondary structure of the LncRNA to be located is .((((.(((. . . .))))))), and the sequence feature information and the structure feature information of the sequence of the LncRNA to be located can be obtained in the manner described above, and input them into the classification prediction model to obtain location prediction values of the LncRNA to be located in each subcell as follows: cytoplasm: 0.757, cell fluid: 0.182, ribosome: 0.001, endoplasmic reticulum: 0.014, exosome: 0.035, and synapse: 0.013. The subcell corresponding to the maximum probability in the location prediction value, i.e., the cytoplasm, is taken as the subcell in which the LncRNA to be located is located, and then the location prediction result is obtained.

In the embodiments provided by the present disclosure, by obtaining the sequence feature information and the structure feature information of the LncRNA to be located; performing the calculation on the sequence feature information and/or the structure feature information on the basis of the attention mechanism to obtain the attention value of the sequence feature information and/or the structure feature information; and inputting the attention value into the classification prediction model to obtain the location prediction result of the LncRNA to be located, so that a correlation between the sequence feature information and the structure feature information of the LncRNA to be located is fully considered when predicting the subcellular location of the LncRNA to be located, thereby improving the accuracy rate of the location prediction of the LncRNA to be located.

Based on the same inventive concept, the embodiments of the present disclosure provide an RNA location prediction apparatus, including: at least one processor, and a memory connected with the at least one processor; and
the memory stores instructions which can be executed by the at least one processor, and the at least one processor performs the RNA location prediction method described above by executing the instructions stored in the memory.

Based on the same inventive concept, an embodiment of the present disclosure further provides a readable storage medium, including:
a memory, configured to store instructions which, when the instructions are executed by a processor, cause an apparatus including the readable storage medium to perform the RNA location prediction method described above.

The readable storage medium may be any available medium or data storage device that can be accessed by a processor, and includes either a volatile memory or a non-volatile memory, or may include both the volatile memory and the non-volatile memory. By way of example and not limitation, the non-volatile memory can include a read-only memory (ROM), a programmable ROM (PROM), an electrically programmable ROM (EPROM), an electrically erasable programmable ROM (EEPROM) or a flash memory, a solid state disk or a solid state drive (SSD), a magnetic memory (e.g., a floppy disk, a hard disk, a magnetic tape, a magneto-optical disc (MO), etc.), and an optical memory (e.g., CD, DVD, BD, HVD, etc.). The volatile memory can include a random access memory (RAM) which can act as an external cache memory. By way of example and not limitation, RAM can be obtained in various forms such as a dynamic random access memory (DRAM), a synchronous dynamic random-access memory (SDRAM), a double data rate SDRAM (DDR SDRAM), an Enhanced Synchronous DRAM (ESDRAM), and a Sync Link DRAM (SLDRAM). The disclosed storage devices in the aspects are intended to include, but are not limited to, these and other suitable types of memories.

Those skilled in the art will appreciate that the embodiments of the present disclosure may be provided as a method, a system or a program product. Accordingly, the embodiments of the present disclosure may take the form of an entirely hardware embodiment, an entirely software embodiment, or an embodiment combining software and hardware. Furthermore, the embodiments of the present disclosure may take the form of a computer program product implemented on one or more readable storage media (including but not limited to a disk memory, CD-ROM, an optical memory, etc.) containing computer/processor available program codes therein.

The embodiments of the present disclosure are described with reference to flow charts and/or block diagrams of a method, an apparatus (a system), and a computer program product according to the embodiments of the present disclosure. It should be understood that each flow and/or block in the flow charts and/or block diagrams and the combination of flows and/or blocks in the flow charts and/or block diagrams can be implemented by computer program instructions. These program instructions can be provided to a general-purpose computer, a dedicated computer, an embedded processor or a processor of other programmable data processing equipment to generate a machine, such that the instructions executed by the computer or the processor of the other programmable data processing equipment generate an apparatus for implementing functions specified in one or more flows of the flow charts and/or one or more blocks of the block diagrams.

These program instructions may also be stored in a readable memory that can direct a computer or other programmable data processing equipment to operate in a particular manner, so that instructions stored in the readable memory are caused to produce an article of manufacture including an instruction device which implements the functions specified in one or more flows of the flowcharts and/or one or more blocks of the block diagrams.

These program instructions may also be loaded on a computer or other programmable data processing equipment, so that a series of operation steps are executed on the computer or other programmable equipment to generate computer/processor implemented processing. Therefore, the instructions executed on the computer/processor or other programmable equipment provide steps for realizing the functions specified in one or more flows of the flow charts and/or one or more blocks of the block diagrams.

Obviously, those skilled in the art can make various changes and modifications to the present disclosure without departing from the spirit and scope of the present disclosure. Thus, if these changes and modifications of the present disclosure fall within the scope of the claims of the present disclosure and their equivalents, the present disclosure is also intended to include these changes and modifications.

1. An RNA location prediction method, comprising:
obtaining sequence feature information and structure feature information of LncRNA to be located;
performing a calculation on the sequence feature information and/or the structure feature information on the basis of an attention mechanism to obtain an attention value of the sequence feature information and/or the structure feature information; and
inputting the attention value into a classification prediction model to obtain a location prediction result of the LncRNA to be located.

2. The method according to claim 1, wherein the obtaining the sequence feature information of the LncRNA to be located comprises:
performing k-mer encoding on a sequence of the LncRNA to be located to obtain at least one k-mer encoding set; wherein k-mer in each k-mer encoding set comprises a same number of bases, and k-mer in different k-mer encoding sets comprises different numbers of bases;
obtaining an embedding vector representation of each k-mer encoding in each k-mer encoding set based on a k-mer pre-training model; and
extracting the sequence feature information of the LncRNA to be located from all embedding vector representations with a convolutional neural network.

3. The method according to claim 2, wherein the performing k-mer encoding on the LncRNA to be located to obtain a plurality of k-mer encoding sets comprises:
sequentially taking continuous k number of bases starting from a first base of the sequence of the LncRNA to be located according to k corresponding to each k-mer encoding set to form one k-mer in a corresponding k-mer encoding set until last k number of bases in the sequence of the LncRNA to be located are taken to form the corresponding k-mer encoding set;
wherein first bases of two adjacent k-mers in a same k-mer encoding set are adjacent in the sequence of the LncRNA, and k is a natural number.

4. The method according to claim 2, wherein a training process of the k-mer pre-training model comprises:
performing k-mer encoding on a sequence of each second LncRNA in a set of second LncRNAs to obtain a plurality of second k-mer encoding sets corresponding to the sequence of each second LncRNA;
taking all the second k-mer encoding sets and a plurality of special characters as a vocabulary for a Bidirectional Encoder Representations from Transformer (BERT) model; and
iteratively training the BERT model with all the second k-mer encoding sets to predict an embedding vector representation of a masked element in the second k-mer encoding sets until a value of a loss function of the BERT model no longer decreases to stop training to obtain the k-mer pre-training model; wherein the BERT model only comprises a MASK-LM task in which elements in the second k-mer encoding sets are partially masked with the special characters, and different second k-mer encoding sets correspond to different masking rates, and the masking rate is a fraction of the special characters in masked second k-mer encoding.

5. The method according to claim 2, wherein the convolutional neural network comprises:
a convolutional layer, comprising a plurality of convolution kernels with different sizes, wherein each convolution kernel is configured to perform a convolution operation on a matrix corresponding to the embedding vector representation; and
a max pooling layer connected with an output end of the convolutional layer, and configured to segment a convolution operation result output by the convolutional layer, and combine an maximum feature value in each of obtained segments into the sequence feature information.

6. The method according to claim 1, wherein the obtaining the structure feature information of the LncRNA to be located comprises:
converting a secondary structure of the LncRNA to be located into a tree structure; and
extracting tree structure features from the tree structure with Tree Lstm as the structure feature information of the LncRNA to be located.

7. The method according to claim 6, wherein the converting the secondary structure of the LncRNA to be located into the tree structure comprises:
taking a base pair in which bases are complementarily paired as a root node of the tree structure, and taking unpaired bases as leaf nodes of a previous node in the tree structure according to a pairing relationship of bases in the secondary structure starting from a first base of the sequence of the LncRNA to be located until a last base of the sequence of the LncRNA to be located to obtain the tree structure; wherein when the first base is unpaired, the root node of the tree structure is empty.

8. The method according to claim 6, wherein the extracting the tree structure features from the tree structure with the Tree Lstm comprises:
taking outputs of all child nodes of a current node currently being processed in the tree structure as an input of the current node starting from a leaf node of the tree structure, and updating a gating vector and a memory component corresponding to the current node according to states of the child nodes until the current node is a root node of the tree structure; wherein input of the leaf node is a corresponding base; and
taking output of the root node as the tree structure features.

9. The method according to claim 1, wherein the performing the calculation on the sequence feature information and/or the structure feature information on the basis of the attention mechanism to obtain the attention value of the sequence feature information and/or the structure feature information comprises:
calculating a relatedness value of a value of each dimension in the sequence feature information with the structure feature information;
performing a normalization calculation on the relatedness value corresponding to each dimension in the sequence feature information to obtain a first attention weight of the value of each dimension in the sequence feature information; and
performing a sum operation on a product of each first attention weight and the structure feature information based on the attention mechanism to obtain an attention value of the sequence feature information relative to the structure feature information.

10. The method according to claim 1, wherein the performing calculation on the sequence feature information and/or the structure feature information on the basis of the attention mechanism to obtain the attention value of the sequence feature information and/or the structure feature information comprises:
calculating a relatedness value of a value of each dimension in the structure feature information with the sequence feature information;
performing a normalization calculation on the relatedness value corresponding to each dimension in the structure feature information to obtain a second attention weight of the value of each dimension in the structure feature information; and
performing a sum operation on a product of each second attention weight and the sequence feature information based on the attention mechanism to obtain an attention value of the structure feature information relative to the sequence feature information.

11. The method according to claim 1, wherein the inputting the attention value into the classification prediction model to obtain the location prediction result of the LncRNA to be located comprises:
inputting the attention value into the classification prediction model to obtain a location prediction value of the LncRNA to be located; and
taking a subcell corresponding to a maximum probability in the location prediction value as a subcell in which the LncRNA to be located is located to obtain the location prediction result.

12. The method according to claim 11, wherein a training process of the classification prediction model comprises:
obtaining first sequence feature information and first structure feature information of each first LncRNA in a tagged set of first LncRNAs;
performing a calculation on the first sequence feature information and the first structure feature information of each first LncRNA based on an attention mechanism to obtain a first attention value;
inputting the first attention value into a classification prediction model to obtain a first location prediction value of a corresponding first LncRNA; and
calculating a loss value based on the first location prediction value and a tag value of the corresponding first LncRNA, and adjusting parameters in the classification prediction model by a back propagation algorithm until the loss value reaches a preset condition to obtain a trained classification prediction model.

13. An RNA location prediction apparatus, comprising:
at least one processor, and
a memory connected with the at least one processor;
wherein the memory stores instructions which can be executed by the at least one processor, and the at least one processor performs the method according to claim 1 by executing the instructions stored in the memory.

14. A readable storage medium, comprising a memory, wherein
the memory is configured to store instructions that, when the instructions are executed by a processor, cause an apparatus comprising the readable storage medium to perform the method according to claim 1.
